(19) United States
(12) Patent Application Publication
(10) Pub. No.: US 2016/0167052 A1
(43) Pub. Date: Jun. 16, 2016

(54) METHOD AND SYSTEM FOR MAINTAINING AND ANALYZING A MICRO-DROPLET

(71) Applicant: Technion Research & Development Foundation Limited, Haifa (IL)

(72) Inventors: Tal Eliezer CARMON, Haifa (IL); Leopoldo Luis Martin RODRIGUEZ, Haifa (IL); Shai MAAYANI, Haifa (IL); Daniel BAR-DAVID, Haifa (IL); Sarah-Tehila ATTAR, Haifa (IL)

(21) Appl. No.: 14/969,141

(22) Filed: Dec. 15, 2015

Related U.S. Application Data
(60) Provisional application No. 62/092,148, filed on Dec. 15, 2014.

(51) Int. Cl. B01L 3/00; G01N 21/552; G01N 33/574

(52) U.S. Cl. CPC B01L 3/502761; B01L 3/502715; G01N 33/574; G01N 21/552; B01L 2200/10; B01L 2200/06; B01L 2300/0663; B01L 2300/0838; B01L 2300/161

(57) ABSTRACT

A microfluidic system comprises a liquid reservoir containing a liquid and having a capillary pipe opening. The microfluidic system also comprises an elongated bar having a proximal end contacting the liquid reservoir in the opening, a distal section at which a micro-droplet is formed, and a middle section continuously maintaining thereon a liquid bridge between the reservoir and the micro-droplet.

### RELATED APPLICATION

This application claims the benefit of priority under 35 USC 119(e) of U.S. Provisional Patent Application No. 60/092,148, filed on Dec. 15, 2014, the contents of which are incorporated herein by reference in their entirety

### FIELD AND BACKGROUND OF THE INVENTION

The present invention, in some embodiments thereof, relates to microfluidics and, more particularly, but not exclusively, to method and system for maintaining and optionally also analyzing a micro-droplet.

A Whispering-Gallery Mode (WGM) is a wave mode that travels around a concave surface. Whispering-gallery light waves have been produced in microscopic glass spheres or toruses, for example, with applications in lasing, opto-mechanical cooling, frequency comb generation and sensing. The WGM light waves are almost perfectly guided round by optical total internal reflection, leading to Q factors in excess of 10¹⁰ being achieved far greater than the best values, about 10⁴, that can be similarly obtained in acoustic cavities. Optical modes in a WGM resonator are inherently lossy due to a mechanism similar to quantum tunneling, material absorption and Rayleigh scattering. However, these loss mechanisms are significantly smaller comparing with other optical resonators such as mirror resonators for example.

Microfluidics is a multidisciplinary field intersecting engineering, physics, chemistry, biochemistry, nanotechnology, and biotechnology. Interest in droplet-based microfluidics systems has been growing substantially in past decades. Micro-droplets offer the feasibility of handling miniature volumes of fluids conveniently.

The information included in this background section of the specification, including any reference cited herein and any description or discussion thereof, is included for technical reference purposes only and is not to be regarded as a subject matter by which the scope of the invention is to be bound.

### SUMMARY OF THE INVENTION

According to an aspect of some embodiments of the present invention there is provided a microfluidic system. The system comprises: a liquid reservoir containing a liquid and having a capillary pipe opening; and an elongated bar having a proximal end in the opening, contacting the liquid reservoir, a distal section at which a micro-droplet is formed, and a middle section continuously maintaining thereon a liquid bridge between the reservoir and the micro-droplet.

According to some embodiments of the invention at least one of the middle and distal sections comprises hydrophilic material for attracting the liquid. According to some embodiments of the invention at least one of the middle and the distal sections comprises gel material for attracting the liquid.

According to some embodiments of the invention the elongated bar comprises a distal end and the system comprises an electric source, a first electrode electrically contacting the liquid and a second electrode positioned adjacent to or in contact with the distal end.

According to some embodiments of the invention the liquid is selected from a group consisting of: organic liquid, water and aqueous solution.

According to some embodiments of the invention the micro-droplet diameter is less than 100 micron.

According to some embodiments of the invention the elongated bar has a generally cylindrical shape body.

According to some embodiments of the invention the elongated bar has a radius between about 1 to 25 microns.

According to an aspect of some embodiments of the present invention there is provided a whispering gallery mode (WGM) resonator system. The system comprises: a solid support holding a micro-droplet of a liquid substance; a wave coupler delivering an input wave to the micro-droplet to excite a WGM therein; and a wave detector system collecting waves following interaction of the waves with the micro-droplet.

According to some embodiments of the invention the input wave is characterized by a wavelength which is equal to a micro-droplet circumference divided by an integer number.

According to some embodiments of the invention the input wave is a coherent light beam provided by a laser source.

According to some embodiments of the invention the input wave is an acoustic wave generated optically or provided by an acoustic wave source.

According to some embodiments of the invention the input wave is a capillary wave generated optically or provided by a capillary wave source.

According to some embodiments of the invention the micro-droplet comprises a bio-analyte further comprising at least one biological organism.

According to some embodiments of the invention the system comprises a signal processor receiving signals pertaining to the interacting wave from the detector, analyzing the signal and providing output characterizing the liquid substance based on the signal.

According to some embodiments of the invention the liquid substance comprises a cell and the signal processor provides output differentiating between cancerous and normal cells.

According to some embodiments of the invention the micro-droplet comprises a chemo-analyte, further comprising at least one dissolved chemical substance.

According to some embodiments of the invention the liquid substance comprises chemical substance and the signal processor provides output indicative of existence or level of the chemical substance in the liquid substance.

According to an aspect of some embodiments of the present invention there is provided a method for stabilizing a micro-droplet. The method comprises: providing an elongated bar having a proximal end, a distal section, and a middle section between the proximal end and the distal section; and establishing contact between a capillary pipe opening of a liquid reservoir containing a liquid substance and the proximal end, to thereby from micro-droplet on the distal section, and to continuously maintain on an external surface of the middle section a liquid bridge between the reservoir and the micro-droplet.

According to some embodiments of the invention the method comprises applying electrical field to the elongated bar to form the micro-droplet.

According to some embodiments of the invention, the method comprises exciting at least one WGM in the micro-droplet by coupling an input wave to the micro-droplet.

According to some embodiments of the invention the wave is an optical wave.

According to some embodiments of the invention the wave is an acoustic wave.

According to some embodiments of the invention the wave is a capillary wave.

According to some embodiments of the invention the method comprises receiving a wave interacting with the micro-droplet, analyzing the wave and generating output characterizing the liquid substance based on the analysis.

According to some embodiments of the invention the liquid substance comprises a cell and the analysis comprises differentiating between cancerous and normal cells.

According to some embodiments of the invention the liquid substance comprises chemical substance and the analysis comprises determining existence or level of the chemical substance in the liquid substance.

According to some embodiments of the invention there is a plurality of bars and the method comprises forming on each bar a micro-droplet comprising at least one biological material.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 illustrates a micro-droplet system, according to certain embodiments;

FIG. 2 illustrates the micro-droplet system in which the droplet is formed by applying voltage, according to certain embodiments;

FIG. 3 illustrates a WGM micro-droplet resonator, according to certain embodiments;

FIG. 4 illustrates generating a capillary mode in a WGM micro-droplet, according to certain embodiments;

FIG. 5 illustrates a flowchart of method for stabilizing a micro-droplet and generating a WGM micro-droplet resonator, according to certain embodiments;

FIG. 6A is an image showing a cylindrical silica stem plasma treated in order to modify its hydrophilicity;

FIG. 6B is a schematic illustration of a system used in experiments performed according to some embodiments of the present invention;

FIG. 6C shows a micrograph of a formed drop, together with its calculated mode (inset);

FIG. 6D shows images captured 5 minutes and 16 hours after drop formation;

FIG. 6E is a graph showing a droplet diameter in microns as a function of the time in minutes;

FIG. 6F shows images of a control droplet formed on a plate, where the images were taken 0 seconds, 1 second and 2 seconds after drop formation;

FIG. 7 is a schematic illustration of an experimental setup used for inducing optical resonances in a micro-droplet;

FIGS. 8A-F show results obtained using the system illustrated in FIG. 7.

### DESCRIPTION OF SPECIFIC EMBODIMENTS OF THE INVENTION

The present invention, in some embodiments thereof, relates to microfluidics and, more particularly, but not exclusively, to method and system for maintaining and optionally also analyzing a micro-droplet.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

It was found by the present inventors that exploiting the benefits of droplet based microfluidics requires a deep understanding of droplet generation, droplet motion, droplet merging, and droplet breakup. For example, free water micro-droplets evaporate in parts of a second in room temperature and pressure and hence are not stable enough for developing biological or chemical sensors in general and more particularly cannot be used to generate a stable WGM resonator with air cladding in room temperature and pressure.

The present inventors successfully formed micro-droplets surrounded by air in a steady state equilibrium at room temperature and pressure and stable WGM micro-droplet resonators.

While an exemplary embodiment may be disclosed with regard to generation of a WGM micro-droplet resonator, it would be readily apparent to one skilled in the art that the teachings are readily adaptable to generation of a sensor comprising of a plurality of WGM micro-droplet resonators. While a single WGM micro-droplet resonator is depicted and described in details herein below, the principles of the present invention are applicable to both a single and a plurality of WGM micro-droplet resonators. As would be understood by one skilled in the art, both types of systems may be utilized in accordance with the present invention.

As used herein, the terms “WGM micro-droplet resonator”, “WGM resonator” and “WGM sensor” are interchangeable and mean a whispering gallery mode resonator generated in a micro-droplet as described herein below.

In the description and claims of the application, each of the words “comprise” “include” and “have”, and forms thereof, are not necessarily limited to members in a list with which the words may be associated.

According to an aspect of some embodiments of the present invention, a microfluidic system, a whispering gallery mode (WGM) resonator system and a method for stabilizing a micro-droplet are provided. The microfluidic system optionally and preferably includes a liquid reservoir for continuously feeding the micro-droplet compensating for spontaneous loss of liquids from the micro-droplet. An elongated bar is in a fluidic contact with the liquid reservoir via a capillary pipe opening, and a droplet is maintained at its distal section.

The inventors have realized that stabilizing and maintaining a micro-droplet in steady state equilibrium in a room temperature and pressure may be achieved by feeding continuously feeding the micro-droplet with liquid from a liquid reservoir via a liquid bridge formed on the elongated bar.

Additionally or alternatively, maintaining the micro-droplet in steady state equilibrium may be achieved by depositing hydrophilic material for attracting the micro-droplet to the elongated bar middle section or modifying the surface to be hydrophilic.

Additionally or alternatively, maintaining the micro-droplet in steady state equilibrium may be achieved by providing at least two electrodes and an electric source, wherein a first electrode is configured to electrically contact the liquid in the reservoir and a second electrode is positioned adjacent to the elongated bar distal end.

Additionally or alternatively, maintaining the micro-droplet in steady state equilibrium may be achieved by providing gel material for attracting the micro-droplet to the elongated bar middle section.

Without wishing to be bound by any specific theory or mechanism of action, the inventors have realized that a WGM micro-droplet resonator may enable detecting of bio-analytes including differentiating between normal and cancerous cells, and/or detecting chemical analytes, due to the micro-droplet micro-volume that may contain a single bio-organism interacting with the WGM light wave for example. The inventors have realized that the WGM light wave is performing typically about 10,000 or more turnarounds inside the WGM micro-droplet resonator before it is emitted and collected by an optical detector, and that the WGM micro-droplet resonator enhances significantly the interaction between the trapped resonance light wave and the micro-droplet content via multiple circulations and significantly improves the detection signal-to-noise ratio.

According to embodiments of the present invention, a sensor is provided comprising a plurality of WGM micro-droplet resonators. Assuming that 1 cell out of 10,000 cells is a cancerous cell, for example, inspecting 10,000 cells in 10,000 WGM micro-droplet resonators, will enable detecting the cancerous cell increasing the signal to noise ratio by a factor of 10,000 since in a single cell per micro-droplet inspection the signal is exposed to noise from one cell trapped in a micro-volume droplet instead of a noise generated by 10,000 cells contained in a larger fluidic volume.

The inventors have realized that the WGM micro-droplet resonator enables the light wave to propagate about 50 meters while circulating the droplet. This is in comparison, for example, with 5 micron propagation in analysis tools such as a Raman spectrometer. Accordingly, an improvement of seven orders of magnitude may be expected in a Raman signal using the present invention WGM micro-droplet resonator comparing to a Raman single obtained by a single pass through a tested sample.

Furthermore, according to embodiments of the present invention, a micro-droplet capillary mode may be excited by a light beam interacting with the WGM light wave resonance increasing the WGM resonator sensitivity to the micro-droplet content and enabling access to the mechanical properties of the analyte including its viscosity, mass density and surface tension for example.

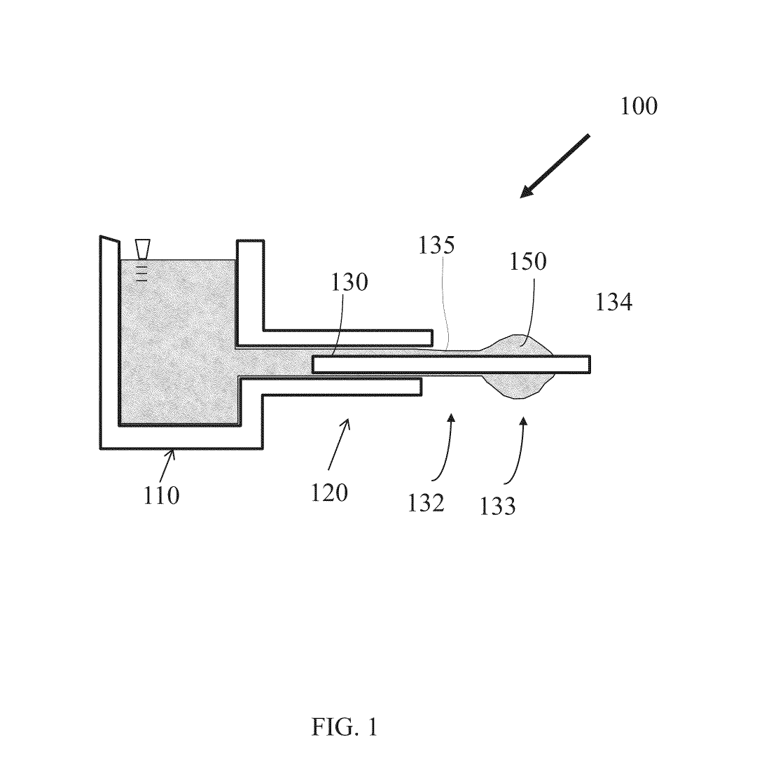

Reference is now made to FIG. 1, which illustrates a microfluidic microfluidic system 100 according to certain embodiments. Microfluidic system 100 may include a liquid reservoir 110 for continuously feeding a micro-droplet 150 compensating for spontaneous loss of liquid (e.g., via evaporation) from micro-droplet 150. Microfluidic system 100 includes an elongated bar having a proximal end 130 in a fluidic contact with the liquid reservoir 110 via a capillary pipe opening 120 and a distal section 133 at which micro-droplet 150 is formed. The elongated bar also a middle section 132 continuously maintaining thereon a liquid bridge 135 between the reservoir and the micro-droplet. The liquid bridge feeds the droplet with liquid and therefore compensates for spontaneous loss of liquid from the micro-droplet (e.g., by evaporation). The elongated bar includes a distal end 134.

Additionally or alternatively, the elongated bar middle section 132 and/or distal section 133 may include hydrophilic material for attracting the micro-droplet 150 to middle 132 and/or distal 133 sections.

Additionally or alternatively, elongated bar middle section 132 and/or distal section 133 may include gel material for attracting the micro-droplet 150 to middle 132 and/or distal 133 sections.

Additionally or alternatively, micro-droplet system 100 may include at least two electrodes and an electric source, wherein a first electrode may be in electric contact with the liquid and a second electrode may be positioned adjacent to the elongated bar distal end.

According to embodiments of the present invention, the micro-droplet liquid may be organic liquid, water and water solution that may include bio-analyte or chemo-analyte.

According to embodiments of the present invention, the micro-droplet 150 is in steady state equilibrium. The diameter of micro-droplet 150 may be less than 4 millimeters and typically less than 100 micron. The steady state equilibrium may be achieved in room temperature (e.g., about 20° C.) and atmospheric pressure (e.g., about 1 atm).

According to embodiments of the present invention, the elongated bar may have a generally cylindrical shape body, wherein the elongated bar radius is preferably, but not necessarily, between about 1 to 25 microns.

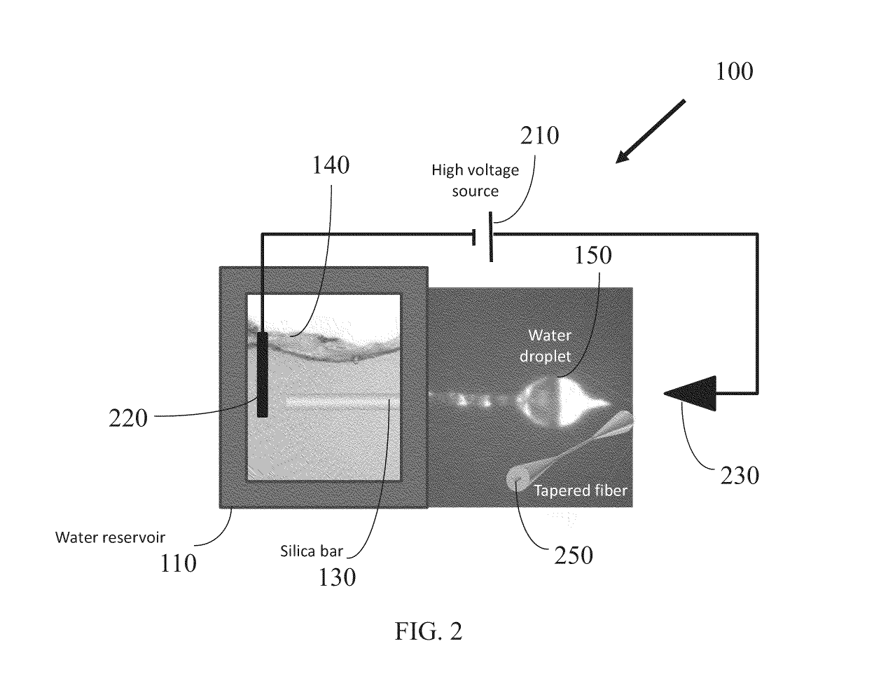

Reference is now made to FIG. 2, which illustrates the microfluidic system 100 in embodiments in which the droplet is formed by applying voltage onto the liquid reservoir 220 and the bar distal end 230, according to certain embodiments. An electric source 210 may be used to attract liquid from reservoir 110 to elongated bar distal section 133 and form the micro-droplet 150. Electrode 220 is in fluidic contact with the liquid in reservoir 110 and electrode 230 is held adjacent to the elongated bar distal end 134 with or without electric contact therewith. The electric field generated by the source and electrodes can be continued after drop formation to maintain the droplet in a steady state equilibrium. Alternatively, as demonstrated in the Example section that follows (see Example 1), the droplet can be maintained in a steady state equilibrium only by means of the liquid bridge formed between the droplet and the liquid in the reservoir. Thus, the present embodiments contemplate a scenario in which once the droplet is formed the electric field is switched off.

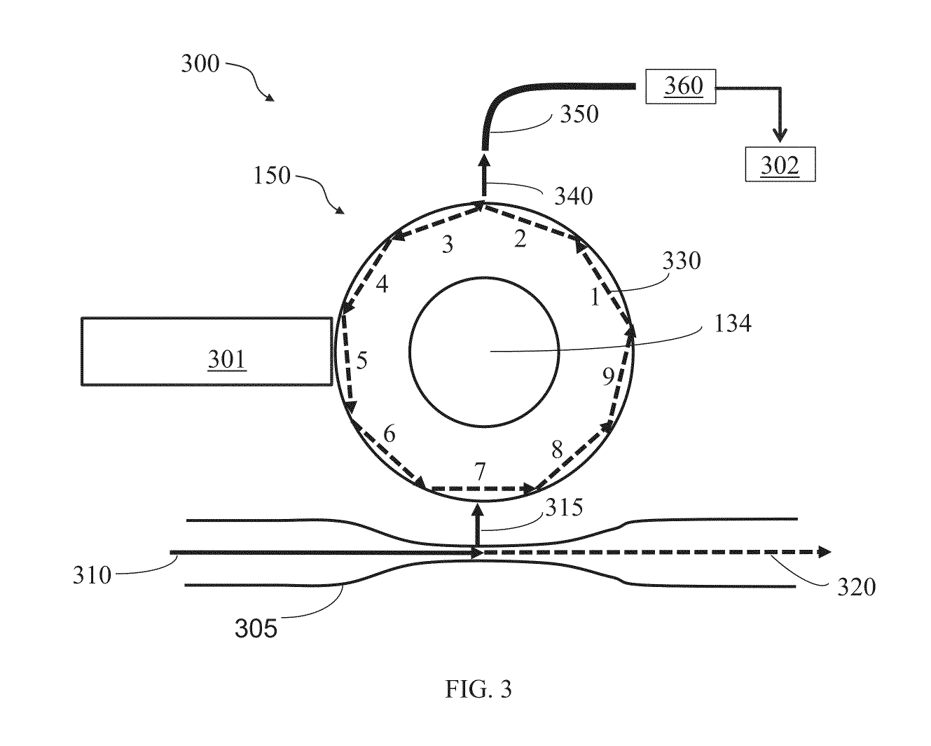

Reference is now made to FIG. 3, which illustrates a WGM resonator system 300, according to certain embodiments. WGM resonator system 300 comprises a solid support (shown as a bar 301, but may have other shapes, e.g., a tapered shape) holding thereon at least one micro-droplet 150 which is typically in a steady state equilibrium. One or more WGMs are excited in micro-droplet 330. The wavelength of WGM 330 is typically equal to micro-droplet 150 circumference divided by an integer number and therefore a resonance is generated by constructive interference in micro-droplet 150. WGM 330 is illustrated by 9 daisy-chained arrows that encircle the WGM micro-droplet 150 circumference. It should be noted that other integer number of wavelength may complete a full circumference of the micro-droplet 150 coherently and may generate the resonance WGM 330.

The WGM 330 can be an optical wave, an acoustic wave and/or a capillary wave, as desired. The WGM 330 can be induced by an input wave 310, which can be a light beam, typically, but not necessarily, a coherent light beam provided by a laser source (not shown). When the input wave 310 is an optical wave, in can induce an optical WGM, an acoustic WGM and/or a capillary WGM. Input light beam 310 may include a plurality of wavelengths where only wavelengths that generate a resonance WGM are coupled efficiently and excite a WGM in micro-droplet 150.

The input wave 310 can alternatively be generated by an acoustic wave source or a capillary wave source (not shown).

WGM 330 is thus excited by coupling 315 input light beam 310 to micro-droplet 150. The coupling can be by a wave coupler 305 (shown as a tapered fiber but be any other type of coupler) micro-droplet.

WGM resonator 300 can optionally and preferably include a wave detector 360 (e.g., a light detector, or an acoustic detector or a capillary wave detector) that collects and guide the wave following interaction of the wave with the droplet. The collected wave can be a wave scattered off the droplet or transmitted through the droplet. In some embodiments of the present invention the wave detector 360 comprises a waveguide 350.

In a representative example, which is not to be considered as limiting, detector 360 is a photomultiplier (PMT) detector. The PMT detector transforms the collected light intensity into an electric signal that may be used to characterize the generated WGM 330.

According to embodiments of the present invention, WGM 330 is responsive to micro-droplet 150 content. Since WGMs are created by constructive wave interference WGMs are sensitive to micro-droplet 150 characteristics, such as the micro-droplet liquid content and hence, according to embodiments of the present invention, the characteristics of the generated WGMs may be used to detect and differentiate between bio-analytes and/or chemo-analytes.

According to embodiments of the present invention, micro-droplet 150 may comprise a bio-analyte further comprising at least one biological organism, where the interaction of the beam of light rotating internally in the stable micro-droplet 150 that comprises the bio-analyte modifies the wavelength of the WGM 330 and the light scattered and collected by the light detector 360.

According to embodiments of the present invention, micro-droplet 150 may comprise a chemo-analyte, further comprising at least one dissolved chemical substance.

According to some embodiments of the present invention system 300 comprises a signal processor 302 that receives signals pertaining to the interacting wave from detector 360, analyzes the signal, and provides output characterizing the liquid substance based on the signal. Signal processor 302 can comprise, for example, a dedicated hardware circuit configured for analyzing the signal and generating the output. For example, the hardware circuit can be configured to differentiate between cancerous and normal cells and provide output indicative whether a cell in the liquid is cancerous or normal. The hardware circuit can also be configured to determine the existence or level of a chemical substance in the liquid and provide output indicative of this existence or level.

According to embodiments of the present invention, a plurality of micro-droplets in a steady state equilibrium in room temperature and pressure, each micro-droplet comprising at least one biological organism and at least one excited WGM, each such WGM resonator may be coupled to an optical fiber and a PMT and used to characterize bio-analytes and/or chemo-analytes with high sensitivity and signal to noise ratio.

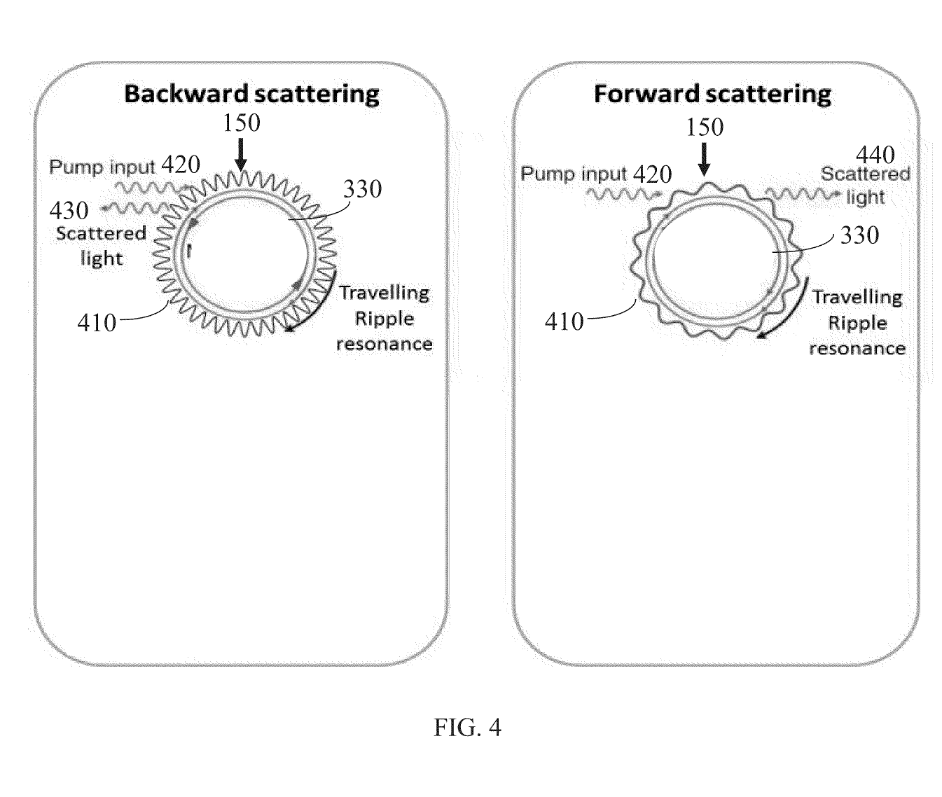

Reference is now made to FIG. 4, which illustrates generating a capillary mode 410 in a WGM micro-droplet 150, according to certain embodiments. Micro-droplet 150 may be excited by a light beam 420 that may be guided by tapered fiber (not shown). Light beam 420 may be configured to excite an optical WGM configured further to excite acoustical or capillary modes 410 in micro-droplet 150 interacting with micro-droplet 330 and its content. Capillary mode 410 is responsive to micro-droplet WGM 330. Light beam input 420 may be configured to interact with capillary mode 410 and the scattered light 430 on the left side or 440 on the right side of FIG. 4, may be collected and guided to a detector, such as, but not limited to, a PMT (not shown), and to a signal processor for analysis. According to embodiments of the present invention, scattered light 430 and 440, emitted by WGM resonator 330 capillary mode 410, may increase the sensitivity of the WGM resonator and may be used to characterize bio-analytes and/or chemo-analytes comprised in micro-droplet 150.

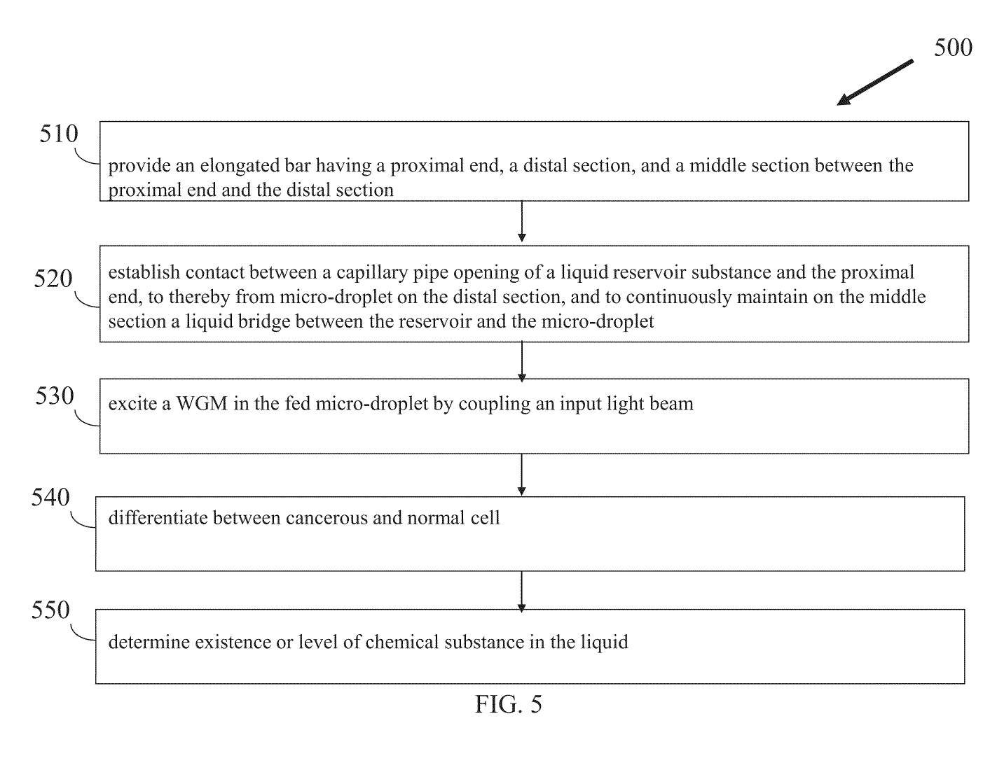

Reference is now made to FIG. 5, which illustrates a flowchart of a method 500 for stabilizing a micro-droplet, according to certain embodiments. Method 500 may include: in stage 510, providing an elongated bar having a proximal end, a distal section, and a middle section between the proximal end and the distal section as further detailed hereinabove; in stage 520, establishing contact between a capillary pipe opening of a liquid reservoir containing a liquid substance and the proximal end, to thereby from micro-droplet on the distal section, and to continuously maintain on the middle section a liquid bridge between the reservoir and the micro-droplet.

Optionally, in stage 530, the method comprises exciting a WGM of the micro-droplet by coupling an input wave (e.g., a light beam or a mechanical vibration) to the micro-droplet. In optional stage 540 the method differentiates between cancerous and normal cell according to at least one characteristic of the excited WGM responsive to the micro-droplet's bio-analyte, by a light detector. In optional stage 550 the method determines existence or level of a chemical substance in the liquid substance.

Advantageously, the above described micro-droplet system is configured to stabilize and maintain a micro-droplet in a steady state equilibrium in room temperature and pressure.

Advantageously, the micro-droplet diameter may be less than 100 micron.

A major advantage of the above described WGM micro-droplet resonator is that light can propagate about 50 meters while circulating in the micro-droplet, providing an improvement of seven orders of magnitude in a Raman signal comparing to a Raman single obtained by a single pass through a tested sample.

Another advantage of the above described micro-droplet system is that a WGM micro-droplet resonator may be generated by coupling light to the micro-droplet and that the WGM light wave is highly sensitive to the content of the micro-droplet content.

Another advantage of the above described WGM micro-droplet resonator is that bio-analytes and/or chemo-analytes may be detected by analyzing scattered light from the WGM micro-droplet resonator.

Another advantage of the above described WGM micro-droplet resonator is that it enhances significantly the interaction between the trapped resonance light wave and the micro-droplet content and significantly improves the detection signal-to-noise ratio due to the small micro-volume of the micro-droplet. It should be noted that the micro-droplet may contain a single bio-organism that interacts with the WGM light wave where the light may perform typically about 10,000 or more turnarounds before it is emitted from the WGM micro-droplet resonator.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

This Example demonstrates application of the technique of the present embodiments for the case in which the liquid is water, and liquid bridge and micro-droplet is surrounded by air.

The advantage of this configuration is that it provides high contrast between air and the water-core for tight confinement (1/Vₘ) with a reduced radiation-loss penalty, where Vₘ is the volume of the optical mode in the micro-droplet. Additionally, the transparency of the air is high so that the water-droplet's optical quality factor, Q, is limited by water absorption only. Still additionally, a substantial portion of the optical intensity, f²(r), in the region-of-interest, r is contained in the water near their interface with air.

This example provides a durable microfluidic device that is surrounded by water walls from most of its sides.

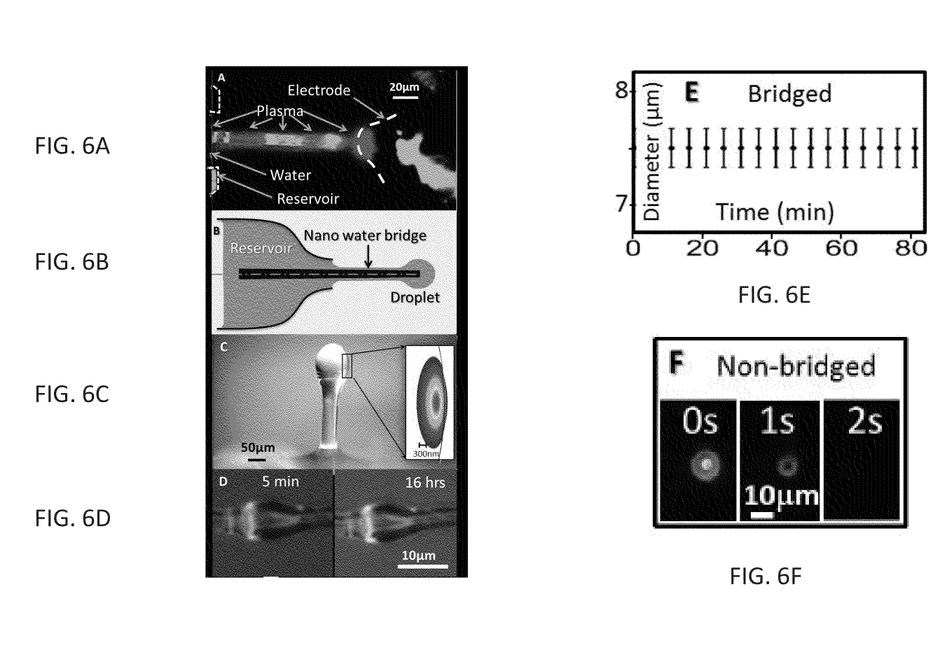

FIG. 6A is an image showing a cylindrical silica stem plasma treated in order to modify its hydrophilicity. FIGS. 6B and 6C describe the droplet sustainer system of the present example. Where, FIG. 6B is a schematic illustration of the system and FIG. 6C shows a micrograph of the formed drop, together with its calculated mode (inset). As shown in FIG. 6B, a nanometric water-bridge extends from the droplet all the way to a practically-unlimited reservoir that feeds it. Total compensation for evaporation is achieved by selecting the hydrophilicity and geometry of the solid surfaces, to enable this bridge, taking into account that water, from energy consideration, prefers to minimize the product of interfacial-tension and area.

In the present Example, a hydrophobic pipette was used to prevent leakage from its small hole. To allow the water inside reaching its thin end, an inner hydrophilic filament was used to enable guidance of water to its narrow end. The wet-plasma treated cylindrical stem (FIG. 6A) at the thin end of the pipette held the micro-droplet while being in fluidic contact with the water bridge.

FIG. 6D shows images captured 5 minutes and 16 hours after drop formation, demonstrating the stability of the micro-drop. Light can circumferentially circulate along an equatorial line of the droplet to form an optical whispering gallery resonance. Water can also surround the full length of the stem to allow the formation of the droplet at its distal end. The existence of the water bridge was confirmed by touching it with a dry object and watching it absorb liquid.

The droplet survived perturbations, including perturbations induced by holding the sustainer by hand and walking with it to another setup. The droplet also survived touching it with the 1 micron tapered fiber. In fact, the taper of the fiber broke in such tests while leaving the droplet intact. This is occurred since the surface tension governs at small scales. The surface tension in the exemplified system was more than 8000 times stronger than gravity as defined by its Eötvös number (see Appendix A), meaning that such droplets withstand high accelerations.

FIG. 6E is a graph showing the droplet diameter in microns as a function of the time in minutes, where the error bars represent microscope resolution, and FIG. 6F shows images of a control droplet formed on a plate, where the images were taken 0 seconds, 1 second and 2 seconds after drop formation. As shown, the droplet formed by the system of the present embodiments is stable (sustained for at least 16 hours, at which time the experiment was ceased by human intervention), while the control droplet evaporated rapidly. It is noted that vertical and horizontal configurations are equivalent, since gravity is negligible when compared to the surface tension.

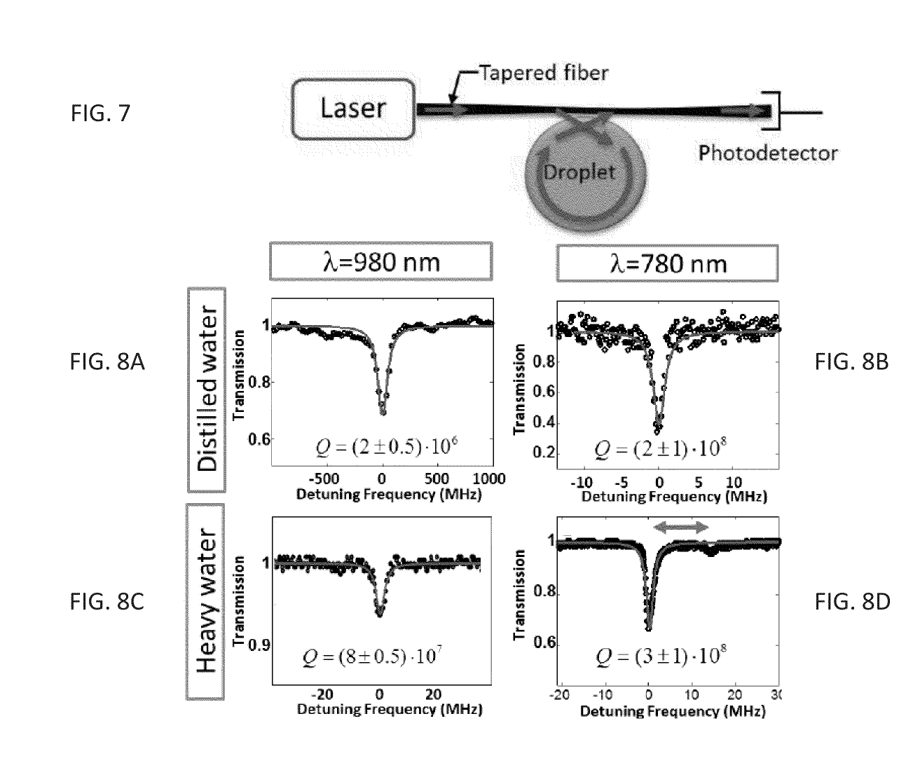

Optical resonances have also been demonstrated in the micro-droplet. The Experimental setup is illustrated in FIG. 7. A tapered fiber was used to evanescently couple light in and out of the droplet that served as an optical resonator. The optical quality factor of the micro-droplet resonator was deduced from the measured optical linewidth (Δλ) by using Q=λ/Δλ. Low optical power was used and the experiment was conducted in an undercoupled regime, so as to prevent linewidth narrowing. Additionally, our the Q measurements were performed at the broaden scan and not at the narrowed one.

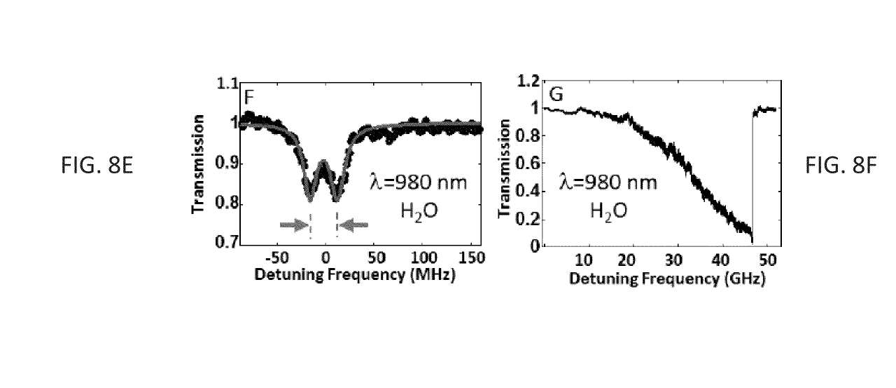

Heavy and distilled water droplets were tested at red and near infrared light to provide Q. The droplet's diameter was between 20 and 40 μm. The transmission as a function of the detuning frequency is shown in FIGS. 8A-F, where FIGS. 8A and 8B show results for distilled water, FIGS. 8C and 8D show results for heavy water, FIGS. 8A and 8C show results for λ=980 nm, FIGS. 8B and 8D show results for λ=780 nm, FIG. 8E demonstrates resolvable splitting between counter-propagating droplet modes at λ=980 nm for distilled water, and FIG. 8F demonstrate coupling efficiency close to 99% at λ=980 nm for distilled water. The red lines in FIGS. 8A-E correspond to a Lorenzian fit.

As shown, both water and heavy water supports an optical finesse above one million. Blue light penetrates water much deeper than red. Thus, 480 nm light can enable at least one order of magnitude improvement in the measured finesse. Still, even at 780 nm, with an optical mode almost completely overlapping with water, the finesse-overlap product is more than 1000 times higher than other devices. As for the interaction of light with thermal capillary waves (FIGS. 8B and 8D) reveals that for a mode as calculated in FIG. 6C (inset) attenuation via scattering from Brownian capillary waves is smaller than 0.0001 cm⁻¹.

The resonance shape that appears in FIG. 8D was analyzed to estimate the droplet's stability from its resonance fluctuations. The resonance shape is 5% distorted when compared to a Lorenzian. Knowing that drifts in the resonance wavelength is proportional to the deviations in cavity radius, about 11 nm/s radius fluctuations were calculated. The stability and linewidth are practically sufficient to resolve splitting between the counter-circulating modes (see arrows in FIGS. 8D and 8E).

Critical coupling was established by bringing the taper closer to the droplet. As a result, transmission at resonance dropped substantially (FIG. 8F), indicating that the coupling efficiency of the exemplified system is nearly 99. Unlike in common solids, the thermal broadening is seen in FIG. 8F while scanning towards the shorter wavelengths. This is because the thermal coefficient of refractive index for water is negative.

### Methods

In the experiments described herein, vertical and horizontal configurations were equivalent, since gravity is negligible when compared with surface tension.

A glass pipette (WPI, TW100-4) was tapered to an inner diameter of 200 μm while heated by a hydrogen flame. A silica stem (Corning, SMF 28) was similarly tapered to form a 10 μm diameter cylinder. The cylindrical stem was introduced into the pipette as illustrated in FIGS. 6A and 6B, and the pipette filled with water. The pipette enacted a water reservoir. The pipette had an inner hydrophilic filament which makes it easier for water to reach its thin side. At the same time, the pipette outer body was made of a less hydrophilic material, which made it difficult for the droplet to spontaneously drip.

Voltage was applied by using two electrodes, one dipped in water (inside the pipette) and the other (platinum electrode) in air. The voltage was increased until obtaining break down to plasma between the electrode and water (at about 1000 V/mm). The voltage was turned off about one second after the plasma was generated. The plasma modified the contact angle between water and silica. Once the concept angle was modified, it was not necessary to repeat the plasma treatment.

In order to pull a water droplet out of the pipette reservoir, low voltage was applied via the same electrodes used to generate the plasma. This time, the voltage was kept below what was needed for generating plasma. The droplet exited out of the pipette all the way to the end of the cylindrical stem so that water was fully covering the cylinder such that free water walls were formed from all sides. The droplet side that was closer to the pipette was water bridged to the pipette reservoir while having a meniscus shape. The voltage was turned off and the drop was sustained at zero voltage as long as long as there was water in the feeding reservoir. Thus, a sustainable droplet was demonstrated by momentarily switching on a relatively low voltage, for about one second.

### Example 2

This Example describes application of the technique of the present embodiments for sensing substance present in water micro-droplet, thereby allowing each liquid droplet to serve as the sample and the sensor at the same time.

Dielectric optical micro-resonators can be used in a wide range of applications, such as, but not limited to, as bio-chemical sensors, micro-lasers and microfluidic elements. The attainable WGMs can have with quality factors of more than 10⁶ or more than 10⁷ or more than 10⁸ or more than 10⁹. In WGM, light travels for a long time along closed paths at the interface between the surface of the resonator and the surrounding environment. In conventional systems, most of the light circulates inside the resonator and only the evanescent-wave tail may interact with the external medium, thereby reducing the effective cavity enhancement. The system of the present embodiments provides a self-sustained volume of liquid where light can be trapped and circulate within the liquid with low loss. Such optical cavities are made of the sample under investigation or their material can be mixed with it. They can also be combined with optical and spectroscopic methods aimed at detection of bio-molecular targets.

The present inventors successfully demonstrated the sensing capabilities of the system of the present embodiments through the quantification of cancer cells and biomarkers in biological fluids, such as blood.

Detection of circulating tumor cells (CTCs) allows serial tumor genotype analysis or assessment of tumor drug sensitivity. CTC evaluation can be used as a diagnostic tool and as a non-invasive source of cancer cells shedding light on the molecular characteristics of the disease through real-time monitoring of the biological changes of the tumor, during its progression and therapy.

The present embodiments provide high-Q optical cavities made of water and combine them with spectroscopic and chemically-selective optical techniques, thereby providing access to the multi-sensory signature of a living cell in its aquatic environment. Fluorescent-protein and molecular-beacon stimulated emission driven by bio-molecular binding can also be used. Spontaneous Raman spectroscopy and stimulated Raman scattering with excitation by frequency-locked lasers can be performed on passive droplets containing the samples. Additionally, metallic nanoparticles can be utilized in the droplet for plasmonic enhancement.

The liquid droplets can be of any type, including, without limitation, surface-standing droplets, pendant droplets, microfluidic droplets, levitating droplets and aerosols. Also contemplated are embodiments in which water is combined with oil and liquid polymer to create a shielding environment and new geometries.

In some embodiments of the present invention a set of independent reactions is performed in a two-dimensional (2D) droplet array to allow executing parallel assays. For example, aerosol droplets can be manipulated by optical tweezers to arrange such a 3D array.

### APPENDIX A

The Eövös number is a dimensionless number that is relevant to our system as it compares gravitational forces to surface tension 33. While at scales longer than a mm, gravity normally dominates, surface tension typically rules when going to the micron scale. For an aquatic system like ours that is having a characteristic size (e.g. diameter) of a 30 μm, the Eövös number suggests that surface tension is 8250 times stronger than gravity. It is therefore that water-walled microfluidics, such as presented here, might function as durable devices even at high accelerations of several Gs.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A microfluidic system, comprising:
a liquid reservoir containing a liquid and having a capillary pipe opening; and
an elongated bar having a proximal end in said opening, contacting said liquid reservoir, a distal section at which a micro-droplet is formed, and a middle section continuously maintaining thereon a liquid bridge between said reservoir and said micro-droplet.

2. The system according to claim 1, wherein at least one of said middle and distal sections comprises hydrophilic material for attracting said liquid.

3. The system according to claim 1, wherein said elongated bar comprises a distal end and the system comprises an electric source, a first electrode electrically contacting said liquid and a second electrode positioned adjacent to or in contact with said distal end.

4. The system according to claim 1, wherein at least one of said middle and said distal sections comprises gel material for attracting said liquid.

5. The system according to claim 1, wherein said liquid is selected from a group consisting of: organic liquid, water and aqueous solution.

6. The system according to claim 1, wherein the micro-droplet diameter is less than 100 micron.

7. The system according to claim 1, wherein said elongated bar has a generally cylindrical shape body.

8. The system according to claim 7, wherein said elongated bar has a radius between about 1 to 25 microns.

9. A whispering gallery mode (WGM) resonator system, comprising:
a solid support holding a micro-droplet of a liquid substance;
a wave coupler delivering an input wave to said micro-droplet to excite a WGM therein; and
a wave detector system collecting waves following interaction of said waves with said micro-droplet.

10. The system according to claim 9, wherein said input wave is characterized by a wavelength which is equal to a micro-droplet circumference divided by an integer number.

11. The system according to claim 9, wherein said input wave is a coherent light beam provided by a laser source.

12. The system according to claim 9, wherein said input wave is an acoustic wave generated optically or provided by an acoustic wave source.

13. The system according to claim 9, wherein said input wave is a capillary wave generated optically or provided by a capillary wave source.

14. The system according to claim 9, wherein said micro-droplet comprises a bio-analyte further comprising at least one biological organism.

15. The system according to claim 9, further comprising a signal processor receiving signals pertaining to said interacting wave from said detector, analyzing said signal and providing output characterizing said liquid substance based on said signal.

16. The system according to claim 15, wherein said liquid substance comprises a cell and said signal processor providing output differentiating between cancerous and normal cells.

17. The system according to claim 9, wherein said micro-droplet comprises a chemo-analyte, further comprising at least one dissolved chemical substance.

18. The system according to claim 15, wherein said liquid substance comprises chemical substance and said signal processor providing output indicative of existence or level of said chemical substance in said liquid substance.

19. The system according to claim 9, wherein said solid support holds a plurality of micro-droplets comprising at least one biological material.

20. A method for stabilizing a micro-droplet, comprising:
providing an elongated bar having a proximal end, a distal section, and a middle section between said proximal end and said distal section; and
establishing contact between a capillary pipe opening of a liquid reservoir containing a liquid substance and said proximal end, to thereby from micro-droplet on said distal section, and to continuously maintain on an external surface of said middle section a liquid bridge between said reservoir and said micro-droplet.

21. The method according to claim 20, further comprising applying electrical field to said elongated bar to form said micro-droplet.

22. The method according to claim 20, comprising exciting at least one WGM in said micro-droplet by coupling an input wave to said micro-droplet.

23. The method according to claim 20, wherein said wave is an optical wave.

24. The method according to claim 20, wherein said wave is an acoustic wave.

25. The method according to claim 20, wherein said wave is a capillary wave.

26. The method according to claim 20, further comprising receiving a wave interacting with said micro-droplet, analyzing said wave and generating output characterizing said liquid substance based on said analysis.

27. The method according to claim 26, wherein said liquid substance comprises a cell and said analysis comprises differentiating between cancerous and normal cells.

28. The method according to claim 26, wherein said liquid substance comprises chemical substance and said analysis comprises determining existence or level of said chemical substance in said liquid substance.

29. The method according to claim 20, wherein there is a plurality of bars and the method comprises forming on each bar a micro-droplet comprising at least one biological material.
